(19) United States
(12) Patent Application Publication
(10) Pub. No.: US 2008/0051685 A1
(43) Pub. Date: Feb. 28, 2008

(54) External fixator linkage

(76) Inventors: Vincent A. Benenati, Dix Hills, NY (US); Gregory S. Ahmad, Brightwaters, NY (US); Michael Vitale, Brooklyn, NY (US)

Correspondence Address: FAY KAPLUN & MARCIN, LLP, 15O BROADWAY, SUITE 702, NEW YORK, NY, 10038, US

(21) Appl. No.: 11/509,461

(22) Filed: Aug. 24, 2006

(51) Int. Cl. A61F 5/00

(52) U.S. Cl. 602/16

(57) ABSTRACT

Described is a linkage for coupling an external fixtaor to a brace supporting an appendage. The linkage comprises, a pair of mounting members, a pair of first elongate members, a pair of position fixing members, a second pair of elongate members and a pair of quick release mechanisms. The mounting members are selectively coupleable to the external fixator. Each of the first elongate members is rotatably coupled to a corresponding one of the mounting members. Each of the position fixing members includes first and second locking channels for slidably receiving a corresponding one of the first elongate members in the first locking channel. Each of the second elongate members is slidably received in a corresponding one of the second locking channels. The quick release mechanisms couple the pair of the first elongate members to the pair of mounting members.

### BACKGROUND

Circular fixation allows precise and dependable correction of limb fracture and deformity (e.g., limb length discrepancy). However, circular fixation also carries a number of risks, most notably joint stiffness and muscular contracture, which may continue even with physical therapy. Correction of muscular contracture may be difficult, if not impossible, without further surgery (e.g., joint mobilization, tendon lengthening). Braces have been developed which allow for controlled motion of a joint adjacent to a limb that is circularly fixated to prevent joint stiffness and muscular contracture. These braces used screwed-on linkages to circular fixators to enhance stability around the joint and restrict motion that may inhibit the healing process.

### SUMMARY OF THE INVENTION

The present invention relates to a linkage for coupling an external fixtaor to a brace supporting an appendage. The linkage comprises, a pair of mounting members, a pair of first elongate members, a pair of position fixing members, a second pair of elongate members and a pair of quick release mechanisms. The mounting members are selectively coupleable to the external fixator. Each of the first elongate members is rotatably coupled to a corresponding one of the mounting members. Each of the position fixing members includes first and second locking channels for slidably receiving a corresponding one of the first elongate members in the first locking channel. Each of the second elongate members is slidably received in a corresponding one of the second locking channels. The quick release mechanisms couple the pair of the first elongate members to the pair of mounting members.

### BRIEF DESCRIPTION OF THE DRAWINGS

FIG. 1 shows an exemplary embodiment of a limb brace system according to the present invention.

FIG. 2 shows an exemplary embodiment of a frame-brace coupling device according to the present invention.

FIG. 3 shows an exemplary embodiment of a linkage for a frame-brace coupling device according to the present invention.

### DETAILED DESCRIPTION

The present invention may be further understood with reference to the following description and the appended drawings, wherein like elements are provided with the same reference numerals. The present invention relates to an external fixator linkage. The exemplary embodiments of the present invention describe a frame-to-brace linkage/coupling device which may detachably couple a circulator fixator of a spatial frame to a limb brace. As will be described further below, uses of the coupling device may allow the brace to be removed for physical therapy, washing the patient, repairing/replacing/cleaning the brace, etc. The coupling device also allows the limb brace to be properly aligned and fitted to the patient and may provide a therapeutic effect to alleviate/prevent muscular contracture, joint stiffness and any other negative effects associated with circular fixation.

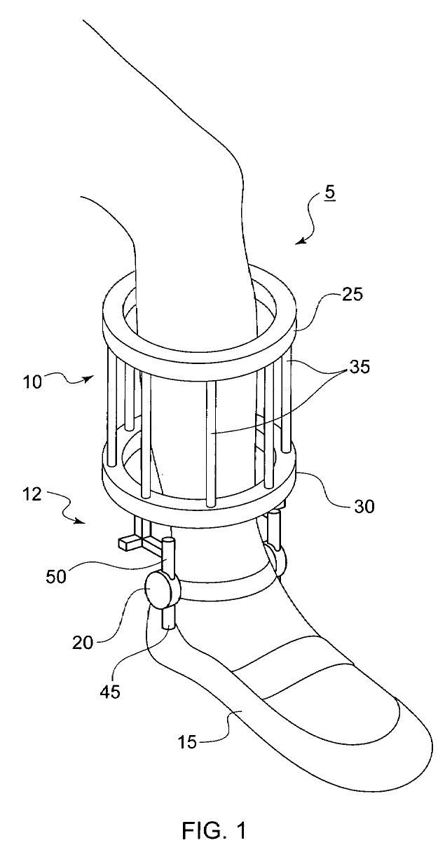

FIG. 1 shows an exemplary embodiment of a limb brace system 5 according to the present invention. The limb brace system 5 may generally comprise an external fixator (e.g., a spatial frame 10) mounted on a limb and a coupling device 12 connecting the spatial frame 10 to a brace 15 via a joint 20. The spatial frame 10 may be a Taylor Spatial Frame or Ilizarov-Taylor Spatial Frame which comprises a proximal circular fixator 25 and a distal circular fixator 30 coupled together via a plurality of struts 35. As is known in the art, the spatial frame 10 may be secured to the limb using, for example, pins, screws, rods, wires, etc. to prevent rotational and/or translational movement of the spatial frame 10 relative to the limb.

The spatial frame 10 may be used to support a fractured limb and/or correct a deformity (e.g., limb length discrepancy) through the concept of callotasis. In the latter instance, a surgical fracture may be created in a long bone in the limb. The bone is allowed to commence healing but is distracted using the spatial frame 10 to regenerate new bone for correcting the deformity, e.g., lengthening the bone. The spatial frame 10 may also be used for a bone transport. In this case, a defect in a long bone may be treated by removing a segment of bone while simultaneously lengthening the bone to replace the removed segment and produce a single bony unit. Regardless of the reason for use, the spatial frame 10 may be mounted over any long bone, e.g., femur, tibia, humerus, ulna.

As shown in FIG. 1, the brace 15 may be a boot worn on the foot when the spatial frame 10 is mounted on the lower leg. However, a structure of the brace 15 may be determined based on the body part on which it will be worn. For example, when the brace 15 to be attached to the lower leg (e.g., the spatial frame 10 is mounted on the upper leg), the brace 15 may be a wrap, a circular band, a cuff, a shell, etc. When the attached to the hand (e.g., the spatial frame 10 is mounted on the lower arm), the brace 15 may also be a glove.

The joint 20 allows the brace 15 to move relative to the spatial frame 10. For example, the joint may comprise 20 a pair of hinges 40 which have distal arms 45 coupled to the brace and proximal arms 50 coupled to the coupling device 12 (coupled to the spatial frame 10). Thus, in the exemplary embodiment shown in FIG. 1, the joint 20 may function substantially similar to an ankle joint allowing extension/flexion of the foot about the joint 20. This embodiment may be similarly implemented over a knee joint. For example, the spatial frame 10 may be mounted on the upper leg (over the femur) and the brace 15 may be worn on the lower leg. The joint 20 may then function substantially similar to a knee joint, allowing extension/flexion of the lower leg about the joint 20. The system 5 may also be implemented on limbs of the upper body. While the joint 20 is shown as a hinge joint only allowing movement in one plane, those of skill in the art that a rotational or sliding joint may be utilized to simulate other body joints and/or other degrees of movement.

In the exemplary embodiment, the hinges 40 of the joint 20 may be configured for operation in a static mode or a dynamic mode. In the static mode, an angle between the proximal arms 45 and the distal arms 50 may be fixed to create a preselected angle between the brace 15 and the spatial frame 10. That is, after a surgical procedure, the brace 15 may be set in a predetermined position creating an initial angle between the spatial frame 10 and the brace 15. Increasing the initial angle to the preselected angle may stretch muscles in the lower leg and foot, alleviating/preventing joint stiffness and muscular contracture.

In the dynamic mode, a continuous pressure may be applied in a predetermined direction to which the patient may apply resistance. For example, if the hinges 40 are configured to apply pressure to cause extension of the foot, the patient may resist the pressure by attempting to flex the foot. The pressure applied by the hinges 40 may be variable, allowing the patient to gradually rebuild and then maintain muscle tone while wearing the spatial frame 10.

Those of skill in the art will understand that various mechanisms may be utilized to implement a dual mode joint as described above. For example, a gearing mechanism having a ratchet may be used to implement the static mode. As the angle between the spatial frame 10 and the brace 15 increases, the ratchet may interlock with a gear to maintain the angle (i.e., resist muscle contracture pulling the brace 15 back to the initial angle). The dynamic mode may be implemented by spring-loading the gearing mechanism and/or adding resistance bands thereto.

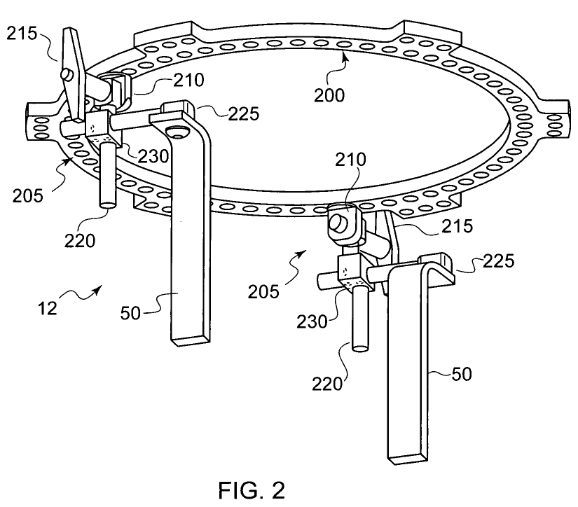

FIG. 2 shows an exemplary embodiment of the coupling device 12 according to the present invention. As noted above, the coupling device 12 may be coupled to the spatial frame 10 and the joint 20. The coupling device 12 allows the joint 20 and the brace 15 to be removed from the patient, enabling the brace 15 to be cleaned, the area previously covered by the joint 20 and the brace 15 to be washed, a physical therapist to easily remove the joint 20 and brace 15, etc. The coupling device 12 also allows a distance between the spatial frame 10 and the joint 20 to be varied for properly aligning the joint 20 with the bodily joint (e.g., ankle, knee, etc.).

The coupling device 12 may include a circular fixator 200 which may be substantially similar to the distal circular fixator 30 on the spatial frame 10. In the exemplary embodiment, the circular fixator 200 is coupled to the distal circular fixator 30 by, for example, mechanical means (e.g., bolts, screws, pins, latches, etc.).

Extending from the circular fixator 200 is a pair of linkages 205. The linkages 205 may be disposed on a circumference of the circular fixator 200 so that they are separated by a distance which corresponds to a distance separating the proximal arms 50 of the joint 20. The linkages 205 may be used to detachably couple the spatial frame 10 to the joint 20. While the exemplary embodiment describes the linkages 205 as attached to the circular fixator 200, those of skill in the art will understand that the linkages 205 may be attached directly to the distal circular fixator 50 of the spatial frame.

In the exemplary embodiment, the linkages 205 include mounting members (e.g., L-brackets 210) that are coupled to the circular fixator 200. Although the L-brackets 210 are shown in FIG. 2 as mechanically coupled to the circular fixator 200 via a mechanical means (e.g., a bolt, a pin, etc.), those of skill in the art will understand that the L-brackets may be electrically or chemically affixed to the circular fixator 200, and that this coupling may be temporary or permanent.

Holes are provided in the L-brackets 210 for receiving quick release mechanisms (e.g., pins 215). In the exemplary embodiment, the pins 215 are removably coupled to the L-brackets 210. For example, each of the pins 215 may comprise a cylindrical portion having a catch which, when the catch is in a retracted position, the cylindrical portion may be passed through the hole. After the cylindrical portion has been passed through the hole, the catch may be released into an expanded position, preventing the cylindrical portion from retreating back through the hole. Control of the catch may be affected using, for example, a button on a face of the pin 215, e.g., depressing the button for the retracted position and releasing the button for the expanded position.

The pins 215 may be used to couple first elongate members 220 to the L-brackets 210. When coupled to the L-brackets 210, the first elongate members 220 may be statically disposed and/or rotatable relative to the L-brackets 210. For example, the first elongate members 220 may be statically disposed after a surgical procedure to ensure that the surgical site heals properly, but a rotational aspect may be gradually introduced to prevent muscular contracture and joint stiffness. The rotational aspect may also be useful for properly aligning the joint 20 with the bodily joint, as explained further below.

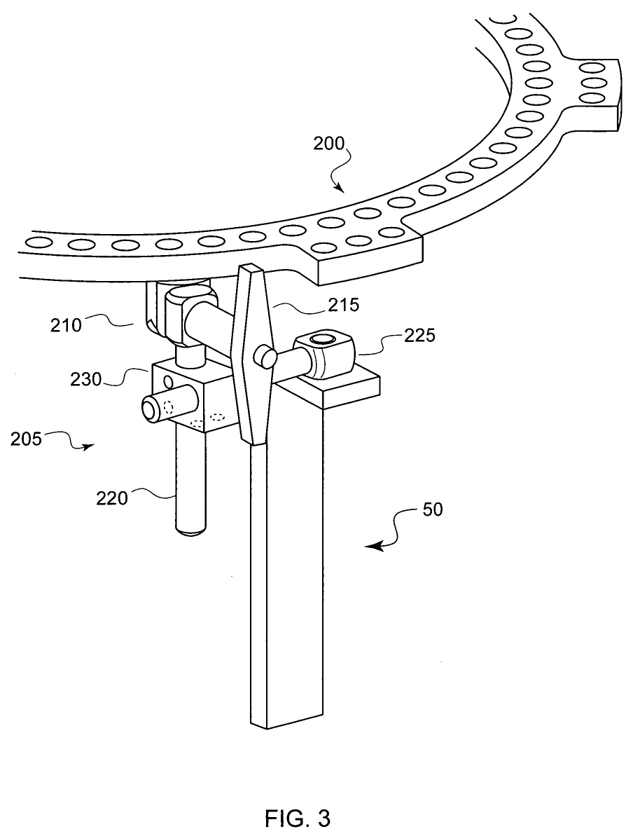

The first elongate members 220 may be coupled to second elongate members 225 via position fixing members (e.g., locking mechanisms 230). As shown more clearly in FIG. 3, the locking mechanism 230 may be implemented as, for example, a sliding block which includes a first channel receiving the first elongate member 220 and a second channel receiving the second elongate member 225. In the exemplary embodiment, the first and second channels may be disposed at a predetermined angle (e.g., substantially perpendicular) relative to each other. A first lock may be disposed on the first channel to lock the first elongate member 220 in a position relative to the block, and a second lock may be disposed on the second channel to lock the second elongate member 225 in a position relative to the block. After the circular fixator 200 of the coupling device 12 is affixed to the distal circular fixator 30 of the spatial frame 10, the first and second elongate members 220, 225 may be moved relative to each other to align the joint 20 with the corresponding bodily joint. The first and second elongate members 220, 225 may include stops to prevent disassociation with the sliding block. When the joint 20 has been properly aligned, the first and second locks (e.g., locking screws) may statically position the first and second elongate members 220, 225 relative to each other, maintaining the joint 20 in its proper alignment (e.g., over the ankle, knee, elbow, etc.). Those of skill in the art will understand that various embodiments of the locking mechanism 230 may be utilized to allow the joint 20 to be properly aligned with the bodily joint.

The second elongate member 225 may be coupled to the proximal arm 50 of the joint 20. The coupling may be affected via mechanical means (e.g., a bolt, screw, etc.) so that the coupling device 12 can be secured to the joint 20.

In an exemplary use of the system 5, the spatial frame 10 may be mounted on the patient following a surgical procedure. For example, after lengthening a bone(s) in the lower leg, the spatial frame 10 may be mounted over the lower leg as is conventionally known in the art. The circular fixator 200 of the coupling device 12 may then be affixed to the distal circular fixator 30 of the spatial frame 10. The brace 15 and the joint 20 may then be mounted on the patient. The second elongate members 225 may then be coupled to the proximal arms 50 of the joint 20, and the joint 20 may be aligned with the ankle joint by positioning the first and second elongate members 220, 225 relative to each other. When the joint 20 has been properly aligned, the first and second elongate members 220, 225 may be locked in their respective positions using the locking mechanism 230.

After the system 5 has been fully mounted on the lower leg and foot, the patient or medical personnel may configure the system 5 for therapeutic operation. As described above, the joints 20 may be configured for the static mode or the dynamic mode to reduce the effects of joint stiffness and muscular contracture. In the static mode, the angle between the spatial frame 10 and the brace 15 may be set to a predetermined value, allowing the muscles, tendons and ligaments of the lower leg to be stretched. In the dynamic mode, the joint 20 may be configured to apply pressure in a predetermined direction (plane, angle, etc.), forcing the brace 15 to extend or flex. In this mode, the patient may resist the pressure strengthen/tone the muscles of the lower leg and foot.

The exemplary embodiments of the present invention allow the patient or medical personnel to remove the brace 15 and the joint 20. For example, when the patient is going to wash, during physical therapy, or when the brace 15 and/or joint 20 needs to be cleaned, repaired, etc., the pins 215 may be removed from the L-brackets 210 on the circular fixator 200. When the pins 215 are removed, the first elongate members 220 are released and the joint 20 and the brace 15 may be removed. Alternatively, the first and/or second locks may be released, allowing the sliding blocks to be removed from the first and/or second elongates members 220, 225, respectively. If the sliding blocks are removed from the first and/or second members, preferably a marking device (e.g., pen, marker, scratch, etc.) is used to mark a position of the first and/or second elongate members 220, 225 relative to sliding blocks.

The present invention has been described with the reference to the above exemplary embodiments. Accordingly, various modifications and changes may be made to the embodiments without departing from the broadest spirit and scope of the present invention as set forth in the claims that follow. The specification and drawings, accordingly, should be regarded in an illustrative rather than restrictive sense.

What is claimed is:

1. A linkage for coupling an external fixtaor to a brace supporting an appendage, the linkage comprising:
a pair of mounting members selectively coupleable to the external fixator;
a pair of first elongate members, each of the first elongate members being rotatably coupled to a corresponding one of the mounting members;
a pair of position fixing members, each of the position fixing members including first and second locking channels, each of the position fixing members slidably receiving a corresponding one of the first elongate members in the first locking channel;
a pair of second elongate members, each of the second elongate members slidably received in a corresponding one of the second locking channels; and
a pair of quick release mechanisms coupling the pair of the first elongate members to the pair of mounting members.

2. The device according to claim 1, wherein the external fixator is a circular fixator.

3. The device according to claim 1, wherein the mounting members are L-brackets.

4. The device according to claim 1, wherein each of the second elongate members includes a connector adapted to couple to a joint connected to the brace.

5. The device according to claim 1, wherein the position fixing members are blocks and the first and second channels are bores formed therethrough.

6. The device according to claim 1, wherein the quick release mechanisms are one of pins and quick release handles.

7. A system, comprising:
a brace supporting an appendage;
a joint coupled to the brace; and
a linkage coupling the joint to a circular fixator via a pair of mounting members selectively coupleable to the circular fixator, the linkage comprising a pair of first elongate members, each of the first elongate members rotatably coupled to a corresponding one of the mounting members, the linkage further comprising a pair of position fixing members, each of the position fixing members including first and second locking channels, each of the position fixing members slidably receiving a corresponding one of the first elongate members in the first locking channel, the linkage further comprising a pair of second elongate members, each of the second elongate members slidably received in a corresponding one of the second locking channels, the linkage further comprising a pair of quick release mechanisms coupling the pair of the first elongate members to the pair of mounting members.

8. The system according to claim 7, wherein the joint is configured to vary an angle between the spatial frame and the brace.

9. The system according to claim 7, wherein the joint simulates movement of a bodily joint between the appendage and a limb on which the circular fixator is mounted.

10. The system according to claim 7, wherein the circular fixator is part of one of a Taylor spatial frame and an Ilizarov-Taylor spatial frame.

11. The system according to claim 7, wherein the brace is one of a boot, a glove, a wrap, a shell, a band and a sleeve.

12. The system according to claim 7, wherein the joint utilizes a static mode to statically maintain the angle between the circular fixator and the brace.

13. The system according to claim 12, wherein the joint includes a ratchet mechanism.

14. The system according to claim 7, wherein the joint utilizes a dynamic mode to apply a pressure forcing the brace to rotate in a predetermined direction around the joint.

15. The system according to claim 14, wherein the joint includes one of a spring-loaded gearing mechanism and a resistance-band mechanism.

16. The system according to claim 7, wherein the joint is a hinge.

17. A device, comprising:
a circular fixator mountable to a spatial frame;
a linkage including a first portion coupled to the circular fixator and a second portion coupled to the first portion and a joint, the joint being coupled to a brace supporting an appendage; and
a pair of quick release mechanisms coupling the first portion to the circular fixator.

18. The device according to claim 17, wherein the first and second portions are first and second elongate members arranged substantially perpendicular to each other and slidably coupled to each other via a locking mechanism.

19. The device according to claim 18, wherein the locking mechanism is a block including a first channel receiving the first elongate member and a second channel receiving the second elongate member, each of the channels including a lock.

20. The device according to claim 17, wherein the spatial frame is a Taylor spatial frame.
